(19) United States
(12) Patent Application Publication
(10) Pub. No.: US 2025/0102532 A1
(43) Pub. Date: Mar. 27, 2025

(54) MODULE FOR MAGNETICALLY EXTRACTING COMPONENTS FROM A SAMPLE

(71) Applicant: BIOMÉRIEUX, Marcy L'Etoile (FR)

(72) Inventors: Edgard MINASSIAN, Lentilly (FR); Jean-Claude RAYMOND, Bessenay (FR)

(73) Assignee: BIOMÉRIEUX, Marcy L'Etoile (FR)

(21) Appl. No.: 18/728,280

(22) PCT Filed: Jan. 17, 2023
(86) PCT No.: PCT/FR2023/000008

§ 371 Date: Jul. 11, 2024

(30) Foreign Application Priority Data
Jan. 18, 2022 (FR) FR2200384

(51) Int. Cl. G01N 35/10; C12M 1/26; C12N 15/10; G01N 35/00

(52) U.S. Cl. CPC G01N 35/10; C12M 33/04; C12N 15/1013; G01N 2035/00465

(57) ABSTRACT

A system for extracting components from a biological sample, the system including at least one extraction platform, a magnetic extraction module including a magnetic portion and a securing portion for securing the module to the extraction platform, the magnetic portion including at least one magnet that is fixedly mounted relative to the securing portion, the extraction platform including a pipetting support that is configured to co-operate with a pipetting device, characterised in that the magnetic portion of the magnetic extraction module has at least one position of use in which the magnetic portion is fixed at least relative to the pipetting support.

The invention relates to a system for magnetically extracting components from a biological sample.

In the present disclosure, “biological sample” means any sample of human origin in which tissues and cells derived from the human body and their derivatives, organs, blood, its components and derived products are defined.

For example, this notably means blood (serum; blood cells (DNA)), urine, stool, saliva, sputum, biopsies (skin, liver, etc.), or sweat, nasal mucous, vaginal fluid, semen, hair, nails, teeth, bones, breast milk, umbilical cord, tissues, etc.

There are several ways of automating the recovery of magnetic particles in biological samples, which employ different magnetic methods for capturing said magnetic particles. However, most of these fail when confronted with the complexity and volume of certain biological samples that need to be processed. One of the means involves the recovery of magnetic particles in sampler cones. The method employs magnetization and demagnetization steps in the cones, requiring the magnet(s) to be moved mechanically past the cones. Such methods require particular and complex kinematics.

### SUMMARY

The present disclosure improves the situation.

To this end, what is proposed is a system for extracting components from a biological sample, comprising at least: an extraction platform, a magnetic extraction module comprising a magnetic portion and a securing portion for securing the module to the extraction platform, said magnetic portion comprising at least one magnet mounted fixedly relative to said securing portion, the extraction platform comprising a pipetting support configured to cooperate with a pipetting device, characterized in that the magnetic portion of the magnetic extraction module has at least one operating position in which the magnetic portion is fixed at least with respect to the pipetting support.

Thanks to the extraction module and its fixed operating position according to the present invention, the manipulation of magnetic particles directly in the sampler cones of the pipetting device is carried out without any movement of the magnet, and this makes it possible to dispense with manual intervention on magnetization management or with the need for complex kinematics dedicated to magnetization management.

The extraction platform according to the present invention also makes it possible to process large volumes of samples by virtue of its ability to circulate the sample past the magnet in said at least one operating position which corresponds to magnetization/demagnetization cycles described below.

According to another aspect, the pipetting device comprises at least one sampler cone, and the magnetic portion of the extraction module is fixed at least in the operating position.

According to another aspect, the securing portion comprises a housing for receiving the extraction platform.

According to another aspect, the module comprises at least one element for holding the extraction platform in said housing.

According to another aspect, the housing comprises a body comprising at least two wraparound walls to wrap around the extraction platform, one of said walls, called the connecting wall, carrying said magnetic portion, said fixed magnet being mounted so as to project relative to said connecting wall, the other wraparound wall being called the end wall.

According to another aspect, the end wall carries said at least one holding element.

According to another aspect, the body comprises a third wraparound wall positioned opposite the end wall.

According to another aspect, the securing portion comprises a body of which one face is shaped to be in contact with the extraction platform.

According to another aspect, the magnetic portion is mounted removably relative to the securing portion.

According to another aspect, the module comprises a unit for holding the magnetic portion, the securing portion and said unit being shaped to allow relative movement of the unit with respect to the securing portion.

According to another aspect, the pipetting device comprises a pipette and a pipette holder, the pipetting support being shaped to receive the pipette holder.

According to another aspect, the module is fixed to the support of the pipette holder by the securing portion.

According to another aspect, the extraction module comprises at least one bar comprising said at least one magnet.

According to another aspect, the system comprises a set of at least two bars, each bar comprising at least one magnet, said at least two bars being arranged parallel to and spaced apart from each other so as to allow the passage of a row of pipette cones of the electronic pipette.

According to another aspect, the module comprises a cross-member for securing each bar.

According to another aspect, the extraction system comprises a plate mounted movably so as to be able to be moved in translation and/or rotation.

Another subject of the invention is a method of extracting components from a sample using a system as described above, comprising a step of moving sampler cones of the pipette against the magnetic portion of the extraction module.

According to another aspect, there is proposed a computer program comprising instructions for implementing all or part of the extraction method as defined herein when this program is executed by a processor.

According to another aspect, there is proposed a non-transient, computer-readable recording medium on which such a program is recorded.

### BRIEF DESCRIPTION OF THE DRAWINGS

Other features, details and advantages will become apparent from reading the detailed description below, and from analyzing the appended drawings, in which:

FIG. 1 illustrates a perspective view of a magnetic extraction module according to a first embodiment of the present invention.

FIG. 2 illustrates a perspective view of an extraction system which may be equipped with the module of FIG. 1.

FIG. 3 illustrates a perspective view of a pipetting system according to the present invention equipped with the module of FIG. 1, in an initial position of a magnetic extraction method, according to a first embodiment of the present invention.

FIG. 4, FIG. 5 and FIG. 6 illustrate respectively a perspective view of the system of FIG. 3 in a sequence of subsequent positions according to the magnetic extraction method of the present invention.

FIG. 7 illustrates a perspective view of a magnetic extraction module according to a second embodiment of the present invention.

FIG. 8 illustrates a perspective view of a pipetting system according to the present invention provided with the module of FIG. 7, in a second embodiment.

FIG. 9 illustrates a perspective rear view of the system of FIG. 8.

FIG. 10 illustrates a perspective view of a magnetic extraction module according to a third embodiment.

FIG. 11 illustrates a perspective view of a pipetting system according to a third embodiment and comprising the module of FIG. 10.

FIG. 12 illustrates a flowchart of the extraction method according to the present invention, implemented particularly in FIGS. 4 to 6.

### DESCRIPTION OF THE EMBODIMENTS

The subject of the invention is an extraction module, referenced 1 in the figures, for magnetically extracting components from a sample, notably a biological sample. The invention also relates to an extraction system for extracting components from a biological sample, also called a pipetting system 100, comprising the extraction module 1 and an extraction platform 101, as will be described in detail below.

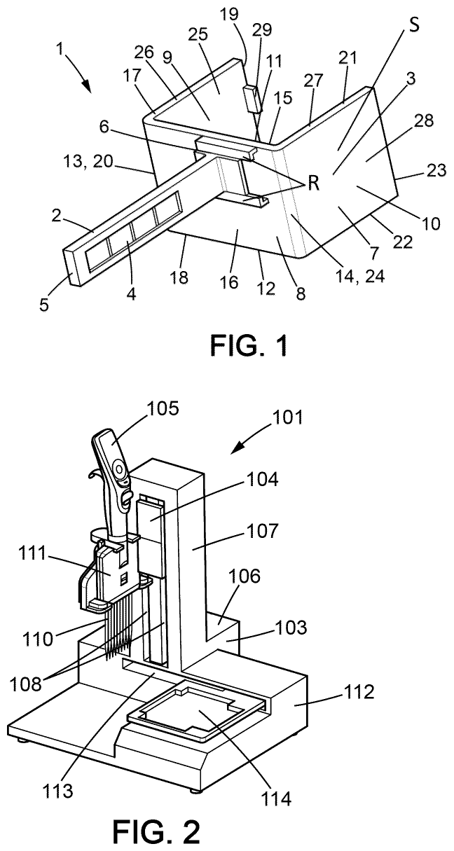

The module 1 according to a first embodiment is described in relation to FIGS. 1 to 6.

With reference more particularly to FIG. 1, the module 1 comprises a magnetic portion 2 and a securing portion S for securing the extraction module to the extraction platform. According to this first embodiment, the securing portion S is a housing 3 for receiving the extraction platform 101, the magnetic portion 2 comprising at least one magnet 4.

In the first embodiment illustrated notably in FIG. 1, the magnetic portion 2 is in the form of a long parallelepipedal bar containing each magnet 4. The bar 2 extends between a free end 5 and an end 6 secured to the housing 3.

The housing 3 comprises a body 7 shaped to fit around a pipetting support of the extraction platform 101, as will be detailed later. Advantageously, the body 7 is provided with at least two wraparound walls to wrap around the extraction platform 101. In the first embodiment illustrated, the body 7 comprises a first, a second and a third wraparound wall, these being respectively referenced 8, 9 and 10.

The first wall 8, also called the connecting wall, carries the magnetic portion 2, the walls 9 and 10, also called the end walls, being disposed one on each side of the wall 8.

As can be seen in FIG. 1, the connecting wall 8 has a generally rectangular shape comprising two long edges 11, 12 and two short edges 13, 14, opposite each other in pairs. The edges 11 to 14 delimit an inner face 15 and an outer face 16.

The inner face 15 is intended to be disposed facing the extraction platform 101 while the outer face 16 is secured to the end 6 of the parallelepipedal bar 2. Preferably, the bar 2 extends in a longitudinal direction orthogonal to the outer face 16.

As can also be seen from FIG. 1, the end walls 9 and 10 each have a generally square shape comprising four edges, respectively referenced 17 to 20 and 21 to 24 and opposite each other in pairs. The edges 17 to 20 delimit an inner face 25 and an outer face 26 of the wall 9. Similarly, the edges 21 to 24 delimit an inner face 27 and an outer face 28 of the wall 10. Of course, the invention is not limited to a square shape of the walls 9 and 10.

In the first embodiment illustrated, the connecting wall 8 and the end wall 9 are secured to one another, the edges 13 and 20 being coincident. The connecting wall 8 and the end wall 10 are also secured to one another, the edges 14 and 24 being coincident.

Preferably, the connecting wall 8 forms with each of the end walls 9, 10 an angle appropriate to the shape of the extraction platform. In FIG. 1, the connecting wall 8 forms with each of the walls 9, 10 an angle of the order of 90°, which gives the housing 3 a U-shaped profile. Thus, the end walls 9 and 10 are disposed facing each other, their inner faces 25 and 27 being positioned facing each other.

The extraction module 1 also comprises a stop 29 mounted on the edge 19 of the wall 9 and a stop (not visible in FIG. 1) mounted on the edge 23 of the wall 10. Each stop provides guidance in the housing 3, as well as holding the extraction module 1 against the extraction platform 101, once the platform has been fitted with the module, as will be described in detail below.

The magnetized bar 2 can be mounted removably from the wall 8, the module 1 comprising grooves R for sliding the bar 2 against the wall 8. The grooves R also allow the insertion of the end 6 against the wall 8. Thus, it is possible to choose a magnet according to the amplitude of the magnetic field and/or according to the types of field lines that it is desired to apply. This optional configuration also makes it possible to remove the magnetized bar for replacement or for maintenance purposes.

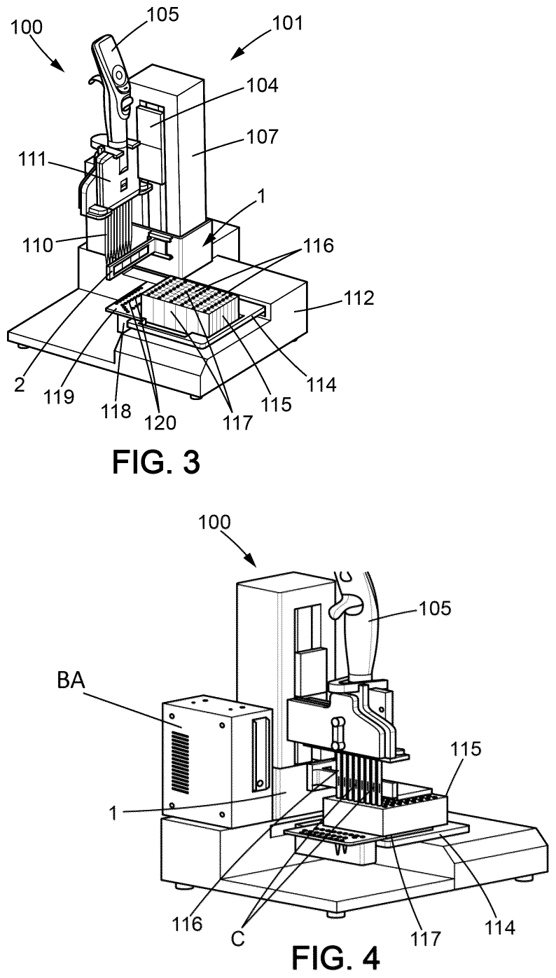

The pipetting system 100 will now be described, notably with reference to FIGS. 2 and 3. As already indicated, the system 100 comprises the module 1 as well as the extraction platform 101.

The extraction system comprises a pipetting support 103, a pipette holder 104 and an electronic pipette 105. The pipette holder 104 and the electronic pipette 105 together form a pipetting device.

The fixed pipetting support 103 comprises a base 106 surmounted by a substantially parallelepipedal block or tower 107. The block 107 is provided with two parallel rails 108 in which the pipetting device can translate. When the system 100 is in its service position, that is to say when the system is in the operating position, the two rails are vertical and extend over at least part of the length of the block 107, which makes it possible to impart to the electronic pipette 105 a vertical translational movement. The block 107 extends the platform from the base 106.

The electronic pipette 105 is housed removably in the pipette holder 104.

As can be seen from FIGS. 2 and 3, the electronic pipette 105 comprises at least one row of sampler cones 110 and a body 111 on which the sampler cones 110 are mounted. An aspiration/discharge circuit (for example a set of pistons actuated by an electric motor) for aspirating liquid from and discharging the liquid into the sampler cones 110, and an electronic control circuit for controlling the aspiration/discharge circuit are integrated into the body 111. The electronic pipette 105 also comprises a human-machine interface notably comprising a screen and a set of buttons for navigation and selection of different pipetting protocols.

The sampler cones 110 have a tubular shape with a longitudinal axis disposed vertically in the service position and an open end of which has a tapered profile or tip. The sampler cones 110 are made of a plastic material, for example polypropylene, which has the effect of making them “transparent” to a magnetic field and allows the capture of magnetic particles, as will be described in greater detail below.

As can also be seen from FIGS. 2 and 3, the extraction platform 101 comprises a base 112 comprising a rail 113 for horizontal translation, in the service position, of a plate 114. The rail 113 extends between an end representing the start-of-travel of the plate 114 and an end representing the end-of-travel, the end-of-travel end being situated closer to the block 107 than the start-of-travel end. The plate 114 is intended to carry a plate 115, illustrated in FIG. 3, also called a microplate. The microplate 115 is provided with a plurality of rows 116 of wells 117 into which the row of sampler cones 110 can be dipped. Each row 116 of wells 117 can receive a liquid solution used during an extraction step carried out by the extraction system 100, as will be described in detail below. The plate 114 allows the microplate 115 to move along the base 112, up to the point where it is vertically in line with the sampler cones 110 of the electronic pipette 105.

As illustrated in FIG. 3, the module 1 is disposed at the base of the block 107, the inner faces 15, 25 and 27 of the wraparound walls 8, 9 and 10 being positioned against a respective face of the block 107. The magnetic bar 2 is placed above the base 112, and above the horizontal rail 113, in front of the row of sampler cones 110 in a direction of approach of the plate 114.

In order to mount the module 1 on the extraction platform 101, the housing 3 is fitted around the block 107 from the top to the bottom, each stop 29 being disposed against a respective edge of the block 107. The stops 29 provide the vertical guidance of the module 1 and then hold the module 1 against the support 103.

As can be seen in FIG. 3, the system 100 also comprises a heating extraction unit 118 with an upper surface 119 pierced by wells 120 allowing the eluate to be collected on a first line, magnetic particles on another line and finally allowing elution on a last line; the compartment 118 is secured to one edge of the plate 114, the upper surface 119 being situated substantially at the same horizontal level as the plate 114.

The system 100 also comprises a power supply unit BA for electrically powering the platform 101. Optionally, the unit BA makes it possible to connect the heating extraction unit 118 and the electronic pipette 105 directly. The unit BA may also comprise a control unit for controlling the electronic pipette 105.

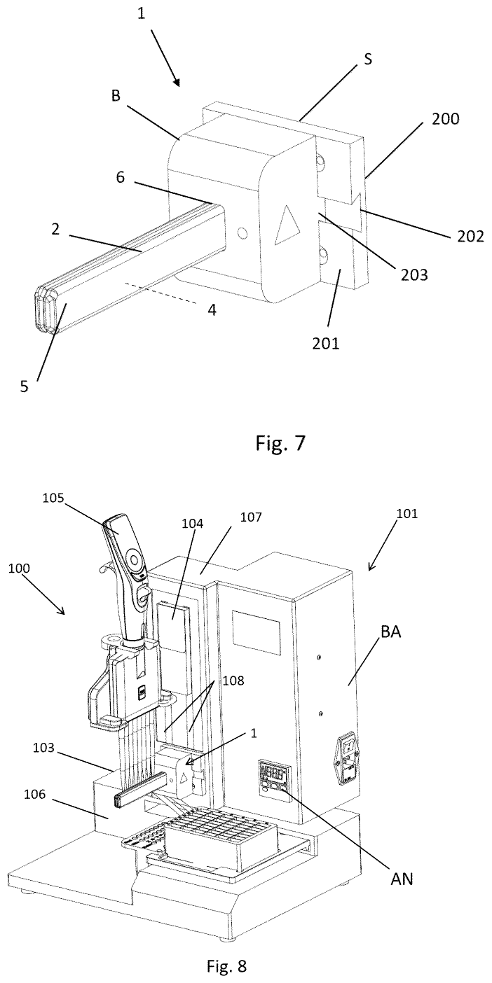

The extraction module 1 according to a second embodiment will now be described in detail, with reference to FIGS. 7 to 9.

With reference more particularly to FIG. 7, the extraction module 1 comprises a magnetic portion 2 and a securing portion S for securing the module to the extraction platform 101, the magnetic portion 2 comprising at least one magnet 4 mounted fixedly relative to the securing portion S.

According to the second embodiment illustrated, the magnetic portion 2 is in the form of a long parallelepipedal bar containing each magnet 4. The bar 2 extends between a free end 5 and an end 6 fitted into a unit B of the module 1. The bar 2 is held fixedly in the unit B, for example by a cleat. It will be noted that the bar 2 and/or the unit B may be mounted removably relative to the securing portion S.

The securing portion S has a generally parallelepipedal shape comprising two opposite faces 200, 201, of which one, 200 (rear face in FIG. 7), is in contact with a pipetting support and the other, 201, is secured to the unit B.

As can be seen from FIG. 7, the face 201 comprises a groove 202 for the sliding of the unit B. The groove 202 advantageously extends in a horizontal direction in the service position. The unit B comprises a wedge 203 in the form of a spline shaped to slide in the groove 202, the groove 202 and the wedge 203 together constituting a dovetail assembly.

According to the second embodiment illustrated, the module 1 does not have a stop for halting the wedge 203 in the groove 202. Nevertheless, it is possible alternatively to provide an end-stop at one or both ends of the groove 202.

According to the second embodiment illustrated, the securing portion S is screwed to the pipetting support 103, the invention of course not being limited to this type of fixing.

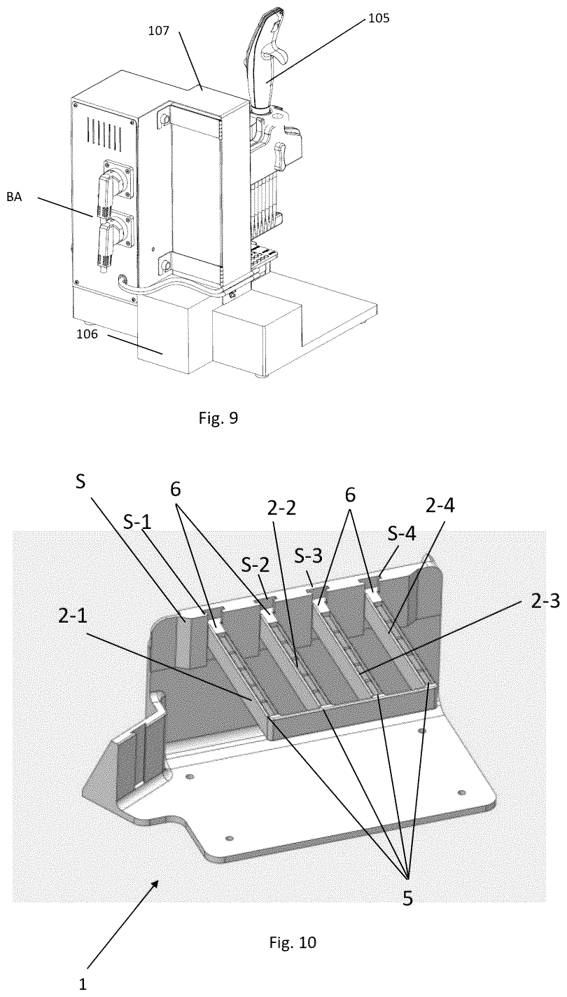

With reference to FIGS. 8 and 9, the extraction platform 101 according to this second embodiment is similar to that of the first embodiment. The system 100 comprises a pipetting support 103, a pipette holder 104 and an electronic pipette 105. The pipette holder 104 and the electronic pipette 105 together form a pipetting device. The fixed pipetting support 103 comprises a base 106 surmounted by a substantially parallelepipedal block 107. The block 107 is provided with two parallel rails 108 in which the pipetting device can translate. When the system 100 is in its service position, that is to say when the system is in the operating position, the two rails are vertical, which makes it possible to impart to the electronic pipette 105 a vertical translational movement. The electronic pipette 105 is housed removably in the pipette holder 104.

As can be seen from FIG. 8, the extraction platform 101 also comprises a tower-shaped power supply unit BA for electrically powering the extraction platform 101. The system 100 also comprises a power supply unit BA for electrically powering the extraction platform 101. Optionally, the unit BA makes it possible to connect a heating extraction unit 118, identical to that presented for the first embodiment, and the electronic pipette 105 directly. The unit BA may also comprise a control unit for controlling the electronic pipette 105.

As can be seen in particular in FIG. 9, the power supply unit BA has a new power supply relay feature so that a heating extraction unit and the electronic pipette can be connected directly to the instrument which is connected to a mains power outlet. Advantageously, the unit BA also controls the temperature of the heating extraction unit 118 via a digital display AN (see FIG. 8).

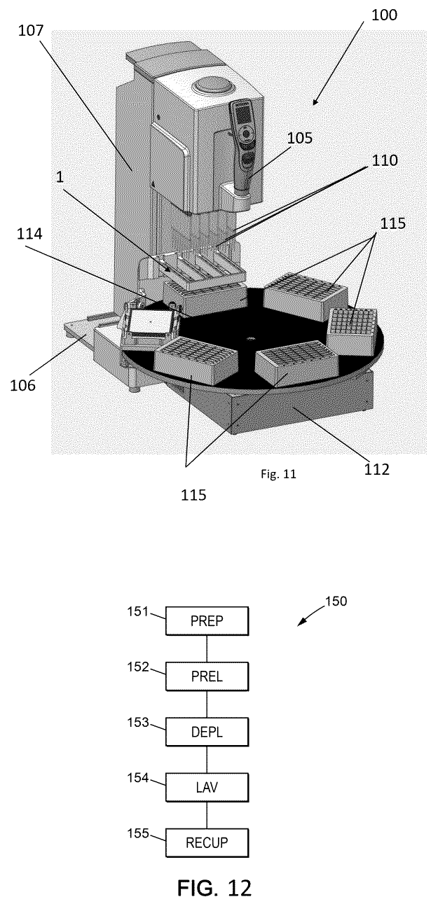

A third embodiment illustrated in FIGS. 10 and 11 will now be described in detail.

According to this third embodiment, the magnetic portion 2 of the extraction module 1 comprises a set E of several (here four) parallelepipedal bars 2-1, 2-2, 2-3, 2-4, each containing at least one magnet 4. The bars 2-1 to 2-4 are arranged parallel to one another and facing one another, two adjacent bars being sufficiently spaced apart to allow a row of sampler cones 110 to pass through. In FIGS. 10 and 11, the bars 2-1 to 2-4 are regularly spaced apart, the invention nevertheless not being limited to this case and a different spacing may be envisioned depending on the type of sampler cones to be analyzed, for example.

Each bar 2-1 to 2-2 is similar to the bar 2 described in relation to the first embodiment, and extends between a first end 5 and a second end 6.

A cross-member T secures the bars 2-1 to 2-4 to one another, each end 5 being fixed to the cross-member T. The other end 6 of each bar 2-1 to 2-2 is secured removably, for preference, to the portion S.

The module 1 according to this third embodiment is able to process several rows of samples at a time, four in FIGS. 10 and 11, and to increase the number of samples that can be processed simultaneously.

As can be seen in FIGS. 10 and 11, the portion S is in the form of a strip provided with four grooves of S-1, S-2, S-3 and S-4 for the sliding of a respective bar 2-1 to 2-4. The grooves extend vertically in the service position.

The extraction system 100 comprises the pipetting support 103, the pipette holder 104 and the electronic pipette 105. The pipette holder 104 and the electronic pipette 105 together form a pipetting device.

The fixed support 103 comprises the base 106 and the tower 107 provided with two parallel rails in which the pipetting device can translate. When the system 100 is in its service position, that is to say when the system is in the operating position, the two rails are vertical, which makes it possible to impart to the pipette 105 a vertical translational movement.

The extraction platform comprises the base 112 surmounted by the horizontal plate 114. According to this third embodiment, the plate 114 is circular and mounted so as to be able to rotate. The plate 114 carries a plurality of microplates 115, which makes it possible to analyze a larger number of samples simultaneously. During the magnetic extraction process, described below, the plate rotates and successively brings the microplates 115 under the electronic pipette 105.

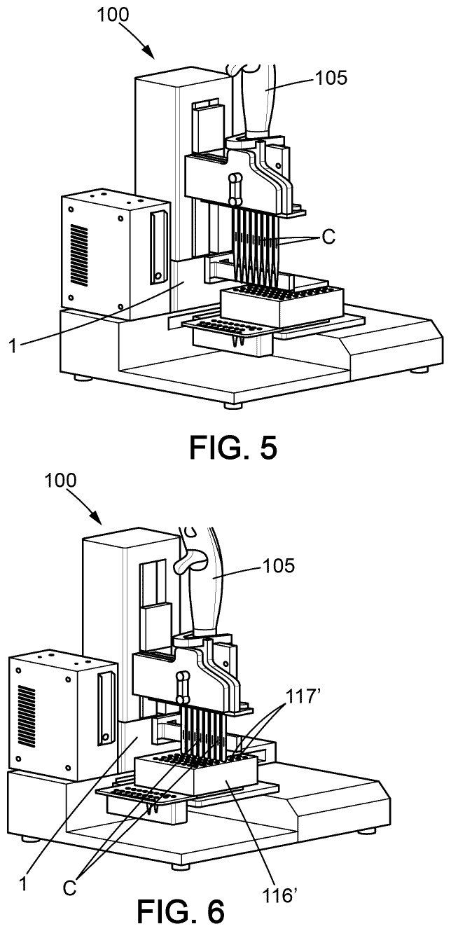

A method of magnetically extracting components from a liquid sample, notably a biological sample, from which it is desired to recover components of interest, is now described in detail, in relation to FIGS. 4 to 6. Of course, the extraction method is also applicable to the second embodiment of FIGS. 7 to 9, as well as to that of FIGS. 10 and 11. The application of the method according to the present invention to the extraction of nucleic acids is described hereinafter, it being understood that the invention is not limited to this application. The method is referenced 150 in FIG. 10 and is applicable whatever the embodiment described above.

A first, preparation, step (151—PREP) consists in mixing the sample with a chemical lysis reagent, and optionally in heating the mixture, and then in introducing magnetic particles into the lysed sample. These are, for example, magnetic silica beads, which are particles having a paramagnetic, ferromagnetic or ferrimagnetic core covered with a silica shell. In a known manner, nucleic acids bind to the surfaces of the magnetic particles, forming with the particles what are known as magnetic pellets. The pellets C are visible in FIGS. 4 to 6.

The preparation also comprises filling at least one row 116 of wells 117 with the solution comprising the lysed sample and the magnetic particles, the microplate 115 being placed on the plate 114. This row 116 is called the analysis row. Optionally, at least one following row 116′, referred to as the washing row, is filled with washing buffer. It will be noted that each liquid level in the rows 116 is situated below the magnetized bar 2 and/or below the field lines of the magnetic field generated by the magnetized bar 2.

In a subsequent step, called sampling (152—PREL), the plate 114 is moved until the analysis row 116 is located under the row of sampler cones 110 and the electronic pipette 105 then executes several aspiration/discharge cycles in the wells 117 of the analysis row 116.

During the aspiration/discharge cycles, the liquid circulates in the vicinity of the magnetized bar 2 and the magnetic pellets are gradually picked up in the sampler cones 110 at the height of the bar 2, as can be seen in FIG. 4.

In order to change between rows of wells (step 153, called displacement step, DEPL), the pipette rises rapidly until the pipetting cones 110 come out of the microplate 115, as illustrated in FIG. 5. The magnetic pellets are then moved away from the field lines of the magnet 4, which causes the magnetic pellets to descend into the cones 110. Nevertheless, the speed of the electronic pipette 105 and that of the displacement of the plate 114 are determined beforehand so that, while the pipette is dipping the cones 110 back into the next row, the magnetic pellets are properly held in the cones 110.

It will be noted that the descent speed of the electronic pipette 105 is determined beforehand so that, during re-descent, the magnetic pellets pass outside the magnetic field of the bar 2, which allows the magnetic pellets to be released into the wells 117 of the washing row 116′.

As can be seen in FIG. 6, the method 150 then comprises a step of washing the magnetic particles, by applying a succession of cycles of aspiration/discharge of the washing buffer, the magnetic pellets once again being gradually picked up in the cones 110 facing the bar 2. This washing step, LAV, is referenced 154 in FIG. 7.

The method optionally includes further washing steps by applying aspiration/discharge cycles in wells of subsequent rows.

The method finally comprises a step of recovering the washed magnetic particles (155—RECUP). During this step, the plate moves to position the cones 110 vertically in line with the magnetic compartment, in the wells of which the magnetic particles are then captured.

It will be noted that, during the aspiration/discharge cycles, that is to say when the system 100 is in the operating position, the magnetic portion is fixed with respect to the pipetting support in each of the embodiments described.

The module 1, when fitted to the extraction platform 101, enables the technician to manipulate the electronic pipette 105 without having to worry about modulating the magnetic field generated by the magnet 4, since it is the movements of the pipetting support 103 and, by extension, of the electronic pipette 105, which ensure this modulation.

The shape of the module 1 ensures simple and secure attachment to the pipetting support 103, by virtue of the open U-shaped profile of the housing and of the stops 29 for holding the block 107 in the housing 3.

The module 1 can be adapted to all dimensions of the block 107 by providing appropriate side-lengths for the connecting wall and end walls 8, 9 and 10.

It will also be noted that the module 1 is secured removably to the block 107, allowing the possibility of potentially using the extraction platform without the module 1, and of making the module 1 interchangeable.

The system 100, when in the service position, makes it possible to implement the extraction method 150 quickly and reliably, in particular by manipulating the magnetic pellets and then recovering the silica particles.

It will be noted that the invention is not limited to the embodiments described. For example, the shape of the housing 3 may differ: the walls 8 to 10 may have other shapes, neither square nor rectangular, depending on the shape of the extraction platform to be equipped. Similarly, it is possible to envision means for securing the module 1 to the pipetting support other than the stops 29. These means may, for example, be slideways carried by the end walls 9, 10. Or again, less advantageously, they may be a set of screws for fixing the housing 1 for the extraction platform. In addition, the module 1 may be equipped with several magnetized bars, regularly or irregularly spaced on the outer face of the connecting wall 8. It is also possible to envision for the magnetic bar to be disposed not orthogonally to the connecting wall 8 but at an angle chosen according to the manipulation to which the magnetic pellets are to be subjected.

1. A system for extracting components from a biological sample, comprising at least:
an extraction platform,
a magnetic extraction module comprising a magnetic portion and a securing portion for securing the module to the extraction platform, the magnetic portion comprising at least one magnet mounted fixedly relative to the securing portion,
the extraction platform comprising a pipetting support configured to cooperate with a pipetting device,
wherein the magnetic portion of the magnetic extraction module has at least one operating position in which the magnetic portion is fixed at least with respect to the pipetting support.

2. The system as claimed in claim 1, wherein the pipetting device comprises at least one sampler cone, the magnetic portion of the extraction module is fixed at least in the operating position.

3. The system as claimed in claim 1, wherein the securing portion comprises a housing for receiving the extraction platform.

4. The system as claimed in claim 3, wherein the housing comprises a body comprising at least two wraparound walls to wrap around the extraction platform, one of said walls, called the connecting wall, carrying the magnetic portion, the fixed magnet being mounted so as to project relative to the connecting wall, the other wraparound wall being called the end wall.

5. The system as claimed in claim 1, wherein the securing portion comprises a body of which one face is shaped to be in contact with the extraction platform.

6. The system as claimed in claim 1, wherein the magnetic portion is mounted removably relative to the securing portion.

7. The system as claimed in claim 6, comprising a unit for holding the magnetic portion, the securing portion and the unit being shaped to allow relative movement of the unit with respect to the securing portion.

8. The system as claimed in claim 1, wherein the pipetting device comprises an electronic pipette and a pipette holder, the pipetting support being shaped to receive the pipette holder.

9. The system as claimed in claim 8, wherein the module is fixed to the pipetting support by the securing portion.

10. The system as claimed in claim 1, wherein the extraction module comprises at least one bar comprising the at least one magnet.

11. The system as claimed in claim 10, comprising a set of at least two bars, each bar comprising at least one magnet, the at least two bars being arranged parallel to and spaced apart from each other so as to allow the passage of a row of pipette cones of the electronic pipette.

12. The system as claimed in claim 11, comprising a cross-member for securing each bar.

13. The system as claimed in claim 1, comprising a plate movably mounted in such a way that it can be moved in translation and/or rotation.

14. A method of extracting components from a sample using a system as claimed in claim 10, comprising a step of moving sampler cones of the pipette against the magnetic portion of the extraction module.
